(19) United States
(12) Patent Application Publication
(10) Pub. No.: US 2004/0127458 A1
(43) Pub. Date: Jul. 1, 2004

(54) Beta-glucan containing compositions, methods for manufacturing beta-glucans, and for manufacturing and using beta-glucans and conjugates thereof as vaccine adjuvants

(76) Inventors: Kenneth W. Hunter, Reno, NV (US); Ruth A. Gault, Reno, NV (US); Frank M. Jordan, Reno, NV (US)

Correspondence Address: THE MATTHEWS FIRM, 1900 WEST LOOP SOUTH, SUITE 1800, HOUSTON, TX, 77027-3224, US

(21) Appl. No.: 10/630,143

(22) Filed: Jul. 30, 2003

Related U.S. Application Data
(63) Continuation-in-part of application No. 09/707,582, filed on Nov. 6, 2000.
(60) Provisional application No. 60/400,377, filed on Aug. 1, 2002.

(51) Int. Cl. A61K 31/715

(52) U.S. Cl. 514/54

(57) ABSTRACT

A microparticulate beta-glucan is used as a vaccine adjuvant for animals and humans, binding to glucan receptors on a variety of phagocytic cells to enhance their immunological functions. The particles contain about 1-10% partially deacetylated N-acetylglucosamine and are predominantly 0.3-3 microns in diameter, preferably 1-2 microns in diameter, to cause the expression of co-stimulatory molecules on antigen presenting cells (APC's).

The microparticle upregulates the expression of the co-stimulatory molecule B7, based upon such microparticles containing beta-(1,3) and beta(1,6) glucan.

### RELATED APPLICATION

This application is a continuation-in-part of U.S. patent application Ser. No. 09/707,582, filed on Nov. 6, 2000, and also claims priority from U.S. Provisional Patent Application Serial No. 60/400,377, filed Aug. 1, 2002.

### TECHNICAL FIELD

The present invention relates generally to an improved method for the preparation of small particle sized glucans. More particularly, the present invention relates to the preparation of small particle sized glucans that modulate immunological activity in humans and animals.

The present invention also generally relates to the immunopharmacologic upregulation of a molecule of a family of B7 molecules to effectuate a costimulatory reaction that allows for an appropriate effector cell immune response.

Additionally, the present invention relates to the use of small particle size glucans as vaccine adjuvants.

### BACKGROUND OF THE INVENTION

Glucans are polymers of glucose. Glucans are commonly found in the cell walls of bacteria, yeast, and various plant species. A common glucan is beta (1,3)-linked glucopyranose (commonly referred to as beta glucan). Other common examples include mixtures of beta-(1,3)-linked glucopyranose with beta-(1,6)-linked glucopyranose. These glucans have been shown to have immunopharmacological activity in humans and animals. More particularly, beta (1,3) glucan has been shown to effect some immune responses.

It is generally recognized that it is desirable to use very small diameter glucan particles to modulate immunological activity, in humans and animals. However, such small particles tend to aggregate, or re-aggregate, upon hydration, or re-hydration, as the case may be, thus reducing or eliminating the desired result.

The re-aggregation and resistance to de-aggregation is accentuated in environments with low pH such as a human digestive tract, such as with a pH of less than 1.0. As the glucans aggregate, or re-aggregate into particles of greater diameter, they appear to pass through an animal or human digestive system without substantially complete absorption.

Thus, there has been a long-felt need for the ability to produce small diameter glucan particles which maintain their particle size without aggregation or re-aggregation upon hydration or re-hydration.

### BRIEF DESCRIPTION OF DRAWINGS

For a further understanding of the nature and objects of the present invention, reference should be made to the following detailed description, taken in conjunction with the accompanying drawings, in which like elements are given the same or analogous reference numbers and wherein;

FIG. 1 is a diagrammatic representation of a unit of beta linked glucopyranose;

FIG. 2 is an illustration of glucan particles in a naturally hydrated state;

FIG. 3 is an illustration of glucan particles and the effects of various methods of preparation;

FIG. 3b is an illustration of a microscopic examination of the effects of dehydration on sonicated glucan;

FIG. 3c is an illustration of a microscopic examination of effects of sonic energy on glucan globules;

FIG. 4 is an illustration of tabular results of a phagocytosis assay;

FIG. 5 is an illustration of tabular results for Nitric Oxide production of a glucan activated macrophage;

FIG. 6 is an immunofluorescence photomicrograph demonstrating the upregulation and cell surface expression of B7.2 on the mouse tumor macrophage cell line P388D1 treated in vitro with a β-1,3-glucan-containing composition;

FIG. 7 is an immunofluorescence photomicrograph demonstrating the upregulation and cell surface expression of B7.2 on mouse peritoneal macrophages treated in vitro with a β-1,3-glucan-containing composition;

FIG. 8 is an immunofluorescence photomicrograph demonstrating the upregulation and cell surface expression of B7.2 on mouse peritoneal macrophages treated in vivo with a β-1,3-glucan-containing composition; and

FIG. 9 is a plot of tabular results of a vaccination study in which a prototypic vaccine antigen (fluorescein isothiocyanate-labeled bovine serum albumin, or FITC-BSA) is administered intradermally to BALB/c mice in either normal saline or as a conjugate with microparticulate glucan (MG).

### GENERAL DESCRIPTION AND PREFERRED MODE FOR CARRYING OUT THE INVENTION

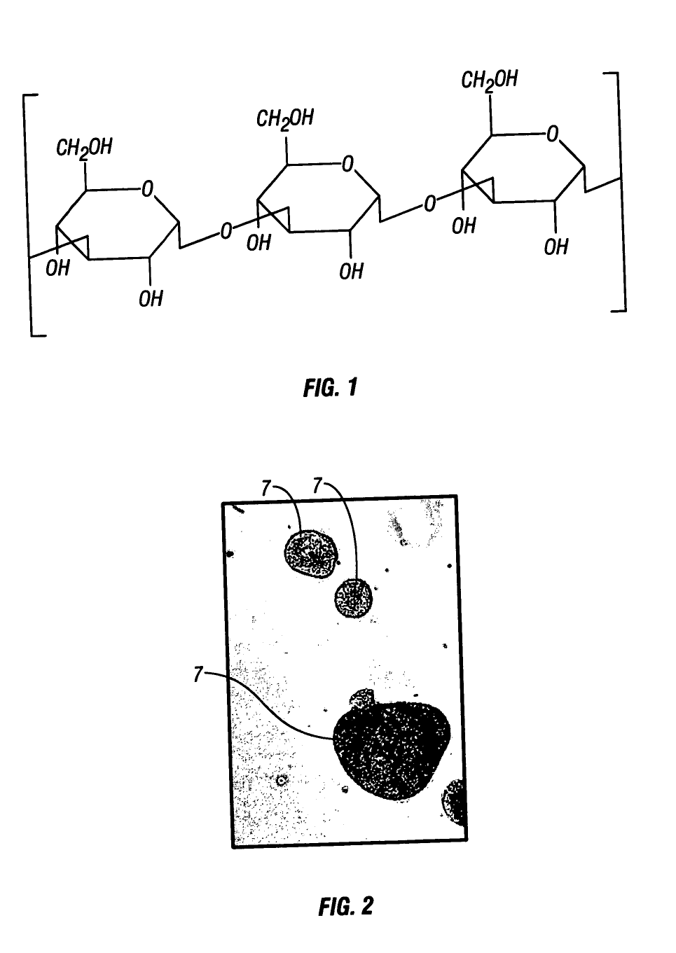

Referring now to FIG. 1, there is illustrated a unit of a beta (1,3) glucan. Generally, under the present method of preparation the resulting size of the glucan polysaccharide can be of any number “n” to produce varying chain lengths.

The glucan containing composition may be made by any means common in the art. A common method of manufacture of a glucan is set out as follows:

1. 0.45 kg of dry Saccharomyces cerevisiae is dispersed in 3.5 L of 0.75 (3%) NaOH

2. Heat to boiling with direct heat. Let stand overnight: decant and discard brown supernatant.

3. Repeat the NaOH digestion (2×)

4. Add 3.5 L of 2.45M HCl to residue. Heat to boiling with direct heat.

5. Let stand overnight: decant and discard light brown supernatant.

6. Repeat the HCl digestion twice, using 1.75M and then 0.94M.

7. To the residue add 2 L distilled water under sufficient pressure to effect mixing.

8. Heat to boiling on hot plate. Let stand overnight: decant and discard supernatant.

9. Repeat the water wash until the residue becomes white and flocculent (20×).

10. To the residue add 1.5 L of abs EtOH. Heat to boiling with direct heat.

11. Let stand overnight: decant and discard yellowish supernatant.

12. Repeat the EtOH extraction until the supernatant becomes colorless (3-4 times).

13. Add 2 L distilled water to the residue under sufficient pressure to achieve mixing.

14. Heat to boiling with direct heat. Let stand overnight: decant and discard supernatant.

15. Repeat the water wash (3×).

16. Pour the washed particulate glucan through a fine silk screen.

17. Shell freeze and lyophilize to dryness.

18. Yield: 2% glucan by volume.

The aforementioned method of preparation of a glucan containing composition is general and it will be understood by those of skill in the art that variations on the aforementioned method will still lie within the scope of the present invention. Further, the resulting glucan composition may be of varying compositions and percentages of glucan. In a most preferred embodiment the resulting glucan solution is about 2% to 5% glucan by volume.

The glucan prepared above exists predominantly in the globule form. It is desirable to reduce the predominant globule size to a range of 0.3-3.0 microns in diameter, preferably to approximately 1-2 microns in diameter. Reducing the size of the glucan globule, for example, down to particles preferably having a range of 1-2 microns in diameter, may be achieved by sonication of a glucan containing composition. In a preferred embodiment a glucan containing composition is first hydrated for a period of at least twelve hours. In a most preferred embodiment the glucan containing composition is hydrated overnight in water.

Then, in a preferred embodiment, a portion of the glucan may be containerized prior to sonication. A preferred embodiment uses an ordinary tray or dish as a container. The container may then be placed in an ultrasonic water bath to dissociate the large globules of glucan. Experimental results have indicated that the size of the container has a direct effect on results of sonication. In a preferred embodiment, a container is selected that allows for 10 to 50 mm space between the container walls and a sonicator probe. However, various other embodiments of the present invention may utilize any variety of containers of varying size.

In a preferred sonication step, the container is sonicated for three-twelve (12) minute intervals with short twelve (12) minute breaks between cycles. In a most preferred embodiment, the container is sonicated for one (1)-twelve (12) minute cycle in an ice bath for cooling the glucan as it is heated during sonication. The short breaks in the cycling are used because the sonication of the glucan generates a considerable amount of heat and cooling of the glucan containing composition is necessary to prevent excessive heating and denaturing or degradation of the product.

In a most preferred embodiment a bench-top sonicator is a BioLogics 300 V/T/Sonic Dismembrator. A preferred embodiment utilizes the settings of the sonicator at 80% power and 80% duty cycle for 12 minute cycles with the container in an ice bath. A preferred probe for use is a 19 mm (¾″) diameter titanium probe. However, other probe may be utilized and still be within the scope of the present invention. A preferred power setting for a one (1) duty cycle sonication is 192 watts for 48 seconds with a 12 second pause at an ultrasonic output of 20 kilocycles per second. Other power settings may be used for sonication, however, the time and number of duty cycles may vary accordingly.

Experimental studies have shown that excessive sonication of the glucan creates heat that may denature the glucan and cause a shortened life of the sonic probe. Accordingly, care should be taken not to over sonicate the glucan and to provide a sufficient time in between cucles to allow the probe to cool. The process of the most preferred embodiment will dissociate substantially all of the glucan globules to the desired diameter, for example, 1-2 microns.

For larger volume small particle glucan production operations, a commercially available sonic dismembrator may be used. Experimental results have shown that the BioLogics, Inc. sonic dismembrator, with a continuous flow chamber, indicated that about 95% of a fully hydrated glucan may be dissociated after one (1) to three (3) treatments at a flow rate of 16 ml/min and 80% power with 12 minutes per treatment. Preferred embodiments of this method utilize one treatment to fully disassociate the glucan.

After sonication, the glucan remains in suspension in an aqueous state for a sufficient period of time for applications requiring suspension of the glucan such as pharmacological applications; including pharmaceutical and pharmacological applications, nutritional applications, and supplementary applications for animals, humans and plants.

In a preferred embodiment, before the initial sonication of the glucan, a percentage gelatin solution, or similar solution, may be added to the glucan solution. In a most preferred embodiment the percentage gelatin solution is a 5% gelatin solution in de-ionized water. In a most preferred embodiment the 5% glucan solution is diluted to about a 2% glucan solution with water and the 5% gelatin solution. Then the glucan may be sonicated as indicated above. In this preferred embodiment the glucan may be utilized wet or dry. A most preferred method for drying the glucan of this embodiment is lyophilizing or freeze drying. A preferred method of freeze drying utilizes an ultralow freezer to freeze the glucan at −80 degrees centigrade. The time required to freeze dry the glucan varies depending upon the amount of glucan, but generally will take between 1 to 2 hours. However, the length of time to freeze dry may vary. The resulting glucan containing composition will dry into a friable, paper-like consistency, and upon re-hydration the glucan will typically disassociate into substantially 1-2 microns in diameter particles.

In another preferred embodiment the wet, gelatinized glucan may be added to a capsule and freeze dried. Upon re-hydration the glucan will de-aggregate into predominantly 1-2 microns in diameter glucan.

In another preferred embodiment, a sugar is added to a sonicated glucan, without the gelatin. In a most preferred embodiment the sugar is maltodextrose. However, other embodiments of the present invention may utilize both a gelatin and a sugar in the glucan. The resulting glucan containing composition may then be placed in a commercially available spray drier for application. A preferred sprayer is the Spray Drying Systems spray drier. A fine, non-aggregated powder, is formed from the spraying. The resulting glucan containing composition, existing substantially as a powder, may then be loaded into capsules, pills or other containers. The powder may also be stored and later re-hydrated for future use.

The preferred spray dryer utilizes an inlet air temperature of 110 to 170 degrees Centigrade, an outlet air temperature of 90 to 120 degrees Centigrade and a feed solids composition of 0.5 to 1.0 percent. However, other settings and spray dryers may be used and be within the scope of the present invention varying the quality of the sprayed glucan. In fact, other settings may be required when using a different spray dryer. The preferred spray dryer produces a finely sprayed glucan powder that does not re-aggregate into glucan globules.

The following examples do not limit the scope of Applicants' invention, but serve as an explanatory tool in the many advantages of Applicants' invention.

### EXAMPLE 1

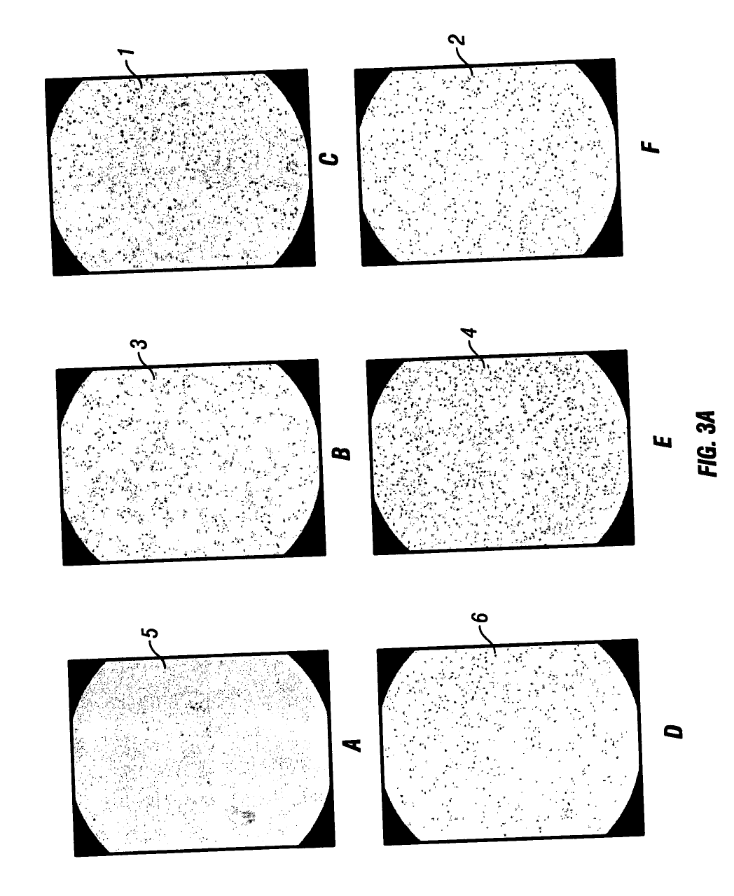

FIG. 2 of the drawings illustrates the morphology of beta-glucan-containing globules of various sizes commonly available on the market. The glucan-containing globules are illustrated in a hydrated state. In the hydrated state, glucan aggregates into globules 7. These glucan globules consist of numerous individual and linked beta glucans. Further, as the glucan aggregates, the size of the glucan globule becomes greater.

### EXAMPLE 2

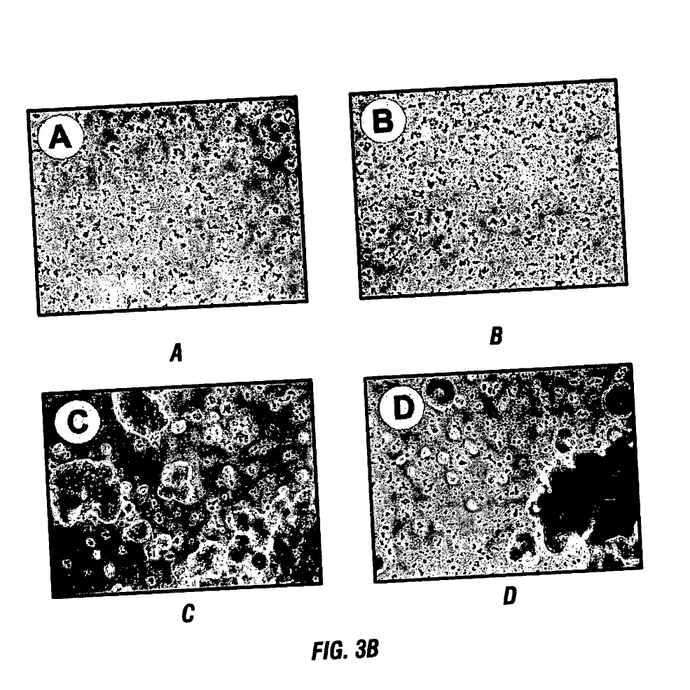

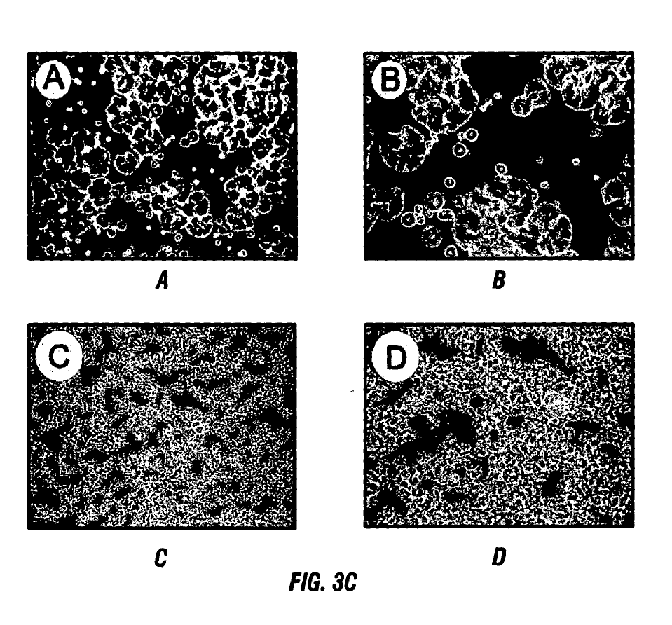

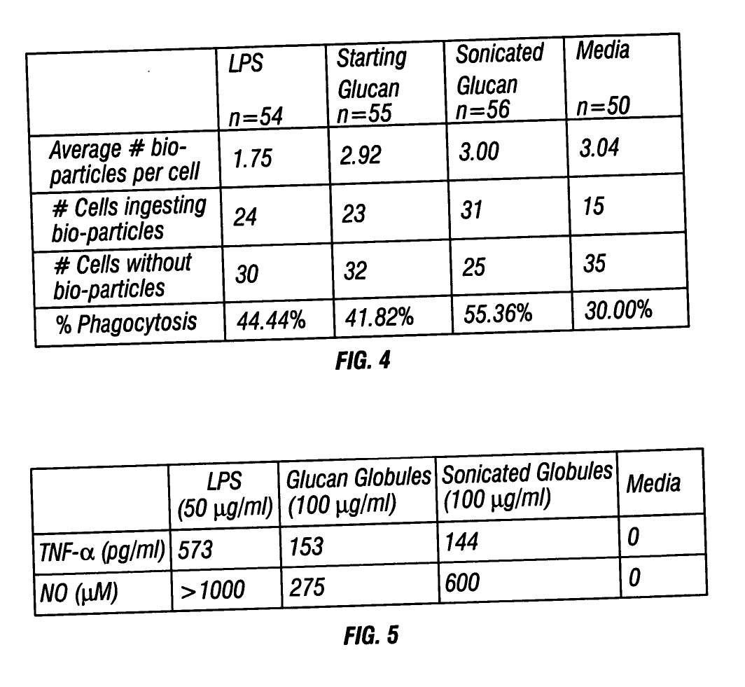

FIG. 3 of the drawing provides an illustration of glucan particle size and the effects of various methods of preparation, demonstrating the reduction in size of the glucan globules upon sonication in accordance with the invention.

Slide C of FIG. 3 is raw glucan at about 200 time magnification. Globule 1 of raw, untreated glucan may be observed. Slide F is a sample of raw glucan at about 200 times magnification that has been ground to a fine ground particle size of the glucan globule 2. It may be observed the glucan globule 2 is generally of smaller size than the glucan globule 1 of Slide C.

Referring now to Slide B of FIG. 3, a sample of raw glucan has been sonicated with a BioLogics V/T Sonic Dismembrator at 80% power and 80% duty cycle for 12 minute cycles in an ice bath, viewed at about 400 times magnification. It may be observed that globule 3 is generally of a smaller globule size than globule 1. It may also be observed that globule 3 is generally more dispersed than globule 1.

Another improvement may be shown in Slide E of FIG. 3, a sample of ground glucan has been sonicated with a BioLogics 300 V/T Sonic Dismembrator at 80% power and 80% duty cycle for 12 minute cycles in an ice bath, viewed at about 400 times magnification. It may be observed that globule 4 is generally of a smaller globule size than globule 1 or globule 2.

Another improvement in the reduction of the globule size may be shown in Slide A of FIG. 3. Slide A is a sample of ground glucan that has been sonicated with a BioLogics 300 V/T Sonic Dismembrator at 80% power and 80% duty cycle for 12 minute cycles in an ice bath, dried and then rehydrated viewed at about 400 times magnification. It may be observed that globule 5 is generally of a smaller globule size than globule 1 or globule 2. It may also be observed that the globule 5 is generally more dispersed than globule 1 or globule 2. It may also be observed that the glucan rehydrated after being dried to contain globule 5 has a globule size generally less than one micron.

Comparable results to that of Slide A were obtained in Slide D of FIG. 3. Slide D is a sample of ground glucan that has been sonicated with a BioLogics V/T/Dismembrator at 80% power and 80% duty cycle for 12 minute cycles in an ice bath, dried and then rehydrated viewed at about 400 times magnification. It may be observed that the globule 6 is generally of a smaller globule size than globule 2 or globule 1.

The experimental results indicate that a small particle glucan of substantially 1-2 microns in particle size may be obtained by sonication without the necessity of grinding, thereby reducing the amount of time required to produce a small particle size glucan.

FIG. 3b of the drawing provides an illustration of a microscopic examination of the effects of dehydration on sonicated glucan and is illustrative of the preferred results after sonication of a glucan. Slide A is a glucan suspension after subjection to three sonication treatments at a flow rate of 16 ml/min and 80% power. The results were taken after drying the glucan and then rehydrating through vortex mixing. Slide B is a glucan suspension after subjection to three sonication treatments at a flow rate of 16 ml/min and 80% power. The results were taken after drying then rehydrated by grinding with a mortar and pestle in de-ionized water. Slide C is a glucan, not sonicated, only suspended in a de-ionized water solution by vortex mixing. Slide D is a glucan, not sonicated, only suspended in de-ionized water after grinding by a mortar and pestle.

As may be observed from FIG. 3b, an illustration is provided of a microscopic examination of the effects of dehydration on sonicated glucan, the resulting glucan is most finely separated in Slide A and in Slide B after both sonication and vortex mixing. Slide C is not finely separated and results in large globules because no sonication was utilized. Slide D results in a more finely separated glucan than Slide C after grinding, but still results in large glucan globules in the absence of sonication.

In FIG. 3c, an illustration is provided of microscopic examination of the effects of sonication on glucan globules, and is demonstrative of the reduction in particle size of glucan globules after sonication. Slide A is a 2% glucan suspension in de-ionized water after vortex mixing viewed at 10×. Slide B is the identical solution of Slide A at 20× magnification. Slide C is Slide A after three treatments of sonication at 16 ml/min at 80% power viewed at 10× magnification. Slide D is the identical solution of Slide C viewed at 40× magnification.

As may be observed from FIG. 3c, an illustration of a microscopic examination of the effects of sonic energy on glucan globules, the glucan globules are reduced to small particle size glucan as a result of sonication.

### EXAMPLE 3

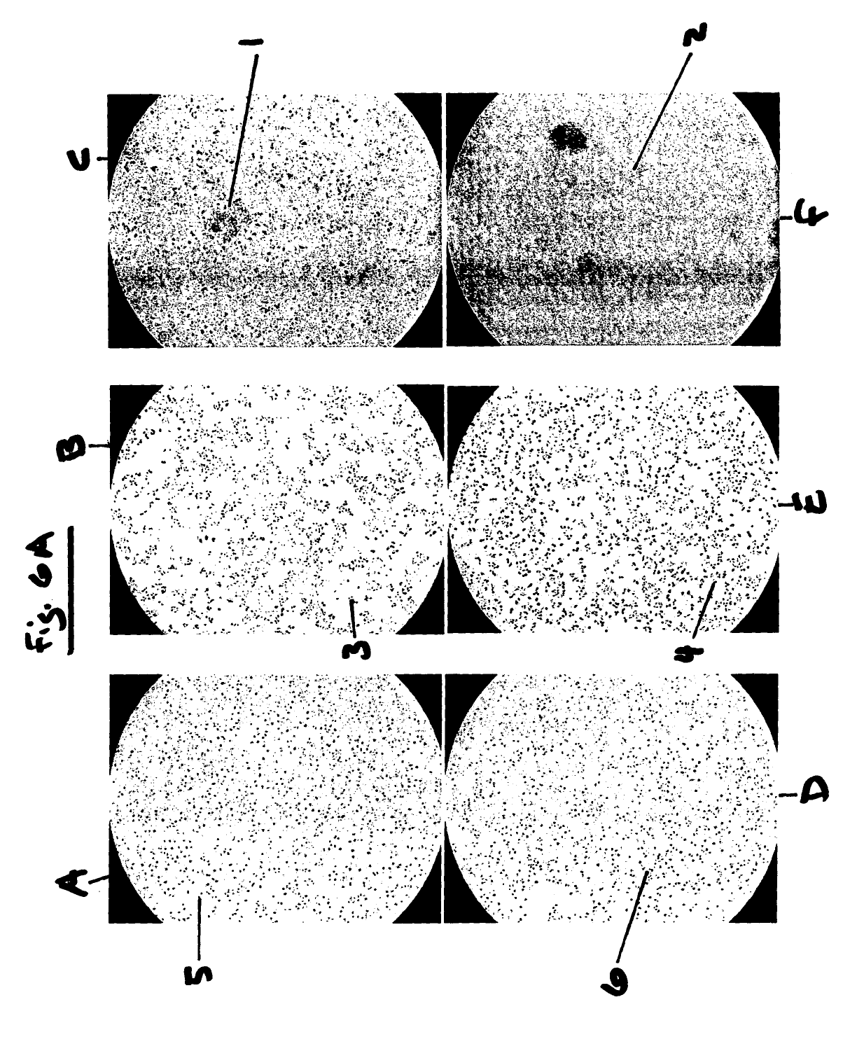

FIG. 4 provides an illustration of a phagocytosis assay and demonstrates the enhanced phagocytosis of the small particle size glucan. The data for FIG. 4, an illustration of a phagocytosis assay, was generated from an assay in which phagocytosis was measured utilizing fluorescent bio-particles. This experiment was conducted to determine the glucan induced macrophage activity. An assay was performed using the ground glucan from FIG. 2, predominantly particle size 1-100 micron in diameter, and another assay was performed using the sonicated beta glucan from FIG. 3, Slide D, predominantly 1-2 microns in diameter, particle size glucan. A bacterium, Staphylococcus aureus, was labeled with a fluorescent marker, fluorescein isothiocyanate (FITC). This dye was chosen because when viewed using fluorescent microscopy the dye emits a yellow-green light.

The labeled cells were mixed with macrophages for about twenty minutes. After incubation, the assays were rinsed with Tryptan Bule, pH 4.4 The acidic solution quenched the fluorescence of FITC, causing the labeled bacterium to no longer emit the yellow-green light. However, the bacterium that have been phagocytised are protected from the quenching and emit the yellow-green light when viewed under the fluorescent microscope.

A comparison of the total number of bacterium ingested in the macrophages of the untreated glucan and treated glucan demonstrate an improved percentage of phagocytosis. As demonstrated by FIG. 4, that average number of bio-particles per cell changed from 2.92 for the raw untreated glucan to 3.00 for the treated glucan.

However, a comparison of the total number of macrophage cells ingesting the bio-particles demonstrated an increase in activity from the untreated macrophage to the treated macrophage. When the total number of macrophages were compared with the total number of macrophages ingesting the bacterium, the percentage phagocytosis was found to be increased from 41.82 percent for the raw untreated glucan to 55.36 percent for the treated glucan. The increased percentage phagocytosis indicates an increase in the activity of the macrophage.

This example was performed with macrophage-like tumor cell lines J774A.1 and P388D1. These cells were allowed to grow on 4 chambered LabTek Tissue Culture Slides to subconfluency. The cells were then exposed to lipopolysaccharide (LPS 50 μg/ml) from Escherichia coli0111:B4, a known activator of macrophages, a solution of glucan globules (100 μg/ml), a solution of DSM-glucan (100 μg/ml) or media. After 1 hour incubation, the stimulant was removed and replaced with growth media. Twenty-four hours post-stimulation the cells were evaluated for activation and a phagocytic index calculated as is demonstrated in FIG. 4.

The greater percentage phagocytosis demonstrates the enhanced activity of the macrophage and the small particle size glucan's ability to activate the immune system.

### EXAMPLE 4

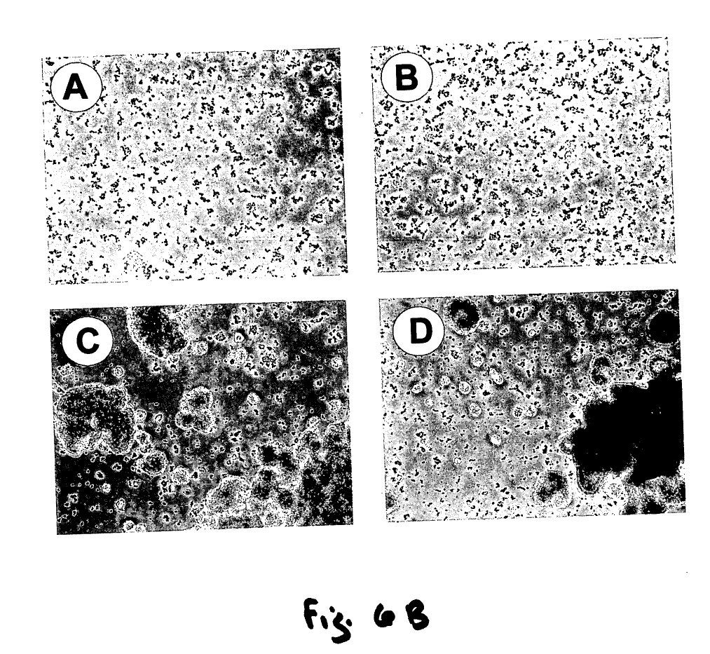

In FIG. 5 of the drawing, there is provided an illustration of tabular results for Nitric Oxide production of glucan activated macrophage and demonstrates the enhanced production of Nitric Oxide, NO, from the untreated glucan to the sonicated glucan. The data demonstrates an approximate factor of two increase in the production of NO from comparison of the untreated glucan to the treated glucan; from 275 μM to 600 μM. The measurement of NO production is indicative of an oxidative burst that kills and/or destroys the ingested microbes and/or particles by the macrophage.

This experiment was performed by measuring NO by antigen capture enzyme immunoassay. Macrophages were stimulated for 1 hour with LPS (50 μg/ml), glucan globules (100 μg/ml), sonicated glucan (100 μg/ml, or media. After stimulation the stimulant was replaced with growth media. Twenty-four hours post-stimulation the culture supernatant was assayed for NO production.

The greater generation and/or production of NO demonstrates the enhanced activity of the macrophage with a small particle size glucan which is indicative of an activity level of an immune system.

Another very important feature of the present invention involves the use of Beta-Glucan containing compositions to potentiate immune responses by upregulating the expressions of costimulatory molecules. As set forth above, glucan-containing compositions are polymers of glucose, and they are commonly found in the cell walls of bacteria, yeast, and various plant species. The glucans can be categorized according to the types of chemical linkages between their glucose monomers, and a common glucan-containing composition is β(1,3)-linked glucopyranose (commonly referred to as β glucan). Other common examples include mixtures of β-(1,3)-linked glucopyranose with β-(1,6)-linked glucopyranose. Glucans bearing these linkages have immunopharmacological activity in animals and humans.

Although there are both soluble and insoluble β-glucan-containing compositions from many microbial and plant sources as substances that can potentiate the immune response to a foreign material such as a microorganism or a tumor, the precise mode of action of these glucan-containing compositions has not been fully elucidated. While macrophage activation is certainly important for innate immunity through the enhanced destruction of pathogenic microorganisms and tumors, the macrophage is also the pivotal cell of the immune system for initiating adaptive immunity. In this role, the macrophage first engulfs foreign material in a process called phagocytosis, then processes these microbial proteins into peptides that are displayed on the macrophage cell surface in association with molecules of the major histocompatibility complex (MHC). Immune cells called T lymphocytes have clonally-derived receptors on their surfaces, and some of these receptors are invested with the ability to bind to a particular peptide so displayed. The end result is the initiation of an immune response involving humoral immunity (antibodies), cell-mediated immunity (killer cells), or both. There is evidence that beta glucan-containing compositions can potentiate both innate and adaptive immunity, but the exact mechanism for this enhancement is not known.

The macrophage and some other cell types have receptors in their surface membranes for β-glucan-containing compositions. When β-glucan-containing compositions interact with the cell surface glucan receptor, the macrophage is activated and becomes capable of direct and indirect killing of the invading pathogen or tumor. However, macrophage activation alone is not responsible for the immunity enhancing effect of glucan. We have discovered a new mode of action of beta glucan-containing compositions that can explain its immunopotentiating effect, and this discovery is a major feature of the present invention.

To understand the importance of this invention, one must understand the mechanism by which the immune response identifies a foreign substance (antigen), and develops a response that can neutralize and/or eliminate the foreign substance before it causes significant morbidity or mortality. The immune system has evolved to discriminate between self and non-self. Non-self could be any of the myriad of potential pathogens for which humans and animals can be hosts, like viruses, bacteria, fungi, and parasites, or the non-self (or altered self) that is represented in the myriad of cancers that can arise from normal cells in an animal or human. The challenge to the immune system is to identify that which is harmful non-self, and to mount a vigorous attack on this foreign material. Extraordinarily potent immune effector mechanisms have evolved, but these effector mechanisms have the potential of causing harm to the animal or human host if they become misdirected to harmless non-self. Unfortunately, as good as the self/non-self discrimination is, the allergic and autoimmune diseases that animals and humans suffer result from errors in discrimination. It is important in the understanding of the present invention to know that the evolution of the immune system included means of avoiding, or at least minimizing, the errors in self/non-self discrimination.

To identify the extraordinary large array of foreign antigens that a human or animal can be exposed, the cells of the immune system evolved a large repertoire of clonally-derived receptors with the ability to bind specifically to the chemicals associated with foreign substances (antigens). To illustrate, we will describe the development of the repertoire of T lymphocyte receptors (TCR). A genetic recombination system has evolved whereby the TCR repertoire is created in the early development of T lymphocytes in the bone marrow. The immature T cells migrate to the thymus where they undergo a two step selection process that first eliminates all T cells bearing receptors that fail to recognize peptides presented on the surface of antigen presenting cells (APC, like macrophages, dendritic cells, and B cells) by major histocompatibility complex (MHC) molecules. Those that pass this test are then retested for strong reactivity against self peptides, thereby eliminating the vast majority of T cells that bear receptors capable of recognizing self peptides. The end result of this selection process is a pool of circulating T cells bearing receptors that, for the most part, will only recognize foreign antigens (central tolerance). Unfortunately, a small number of the T cells will bear receptors that can recognize certain self peptides (e.g., peptides from proteins not found in the thymus), and therefore have the potential of attacking certain self cells and causing immunopathology. To further protect against autoimmune responses, a mechanism known as peripheral tolerance has evolved. The understanding of peripheral tolerance is key to understanding the subject of the present invention.

If naive, but potentially autoreactive, T cells could bind to normal cells via their TCR and be induced to proliferate and differentiate into armed effector cells, these T cells could attack and kill self tissues causing disease and possibly death. To further protect against this potentiality, the immune system evolved a process whereby a naive T cell must receive two signals in order to proliferate and differentiate into an armed effector cell. The first signal is delivered by the TCR binding to its target peptide presented to it by an MHC molecule on the surface of an APC. In the case of the T cell bearing a TCR that recognizes a self peptide, if the first signal was sufficient to allow it to become an armed effector cell, then interaction of that cell with normal tissue could result in the development of a clone of autoreactive effector T cells that would attack the normal tissue. However, the mechanism of peripheral tolerance has evolved to prevent this from happening. A second signal (costimulatory signal) is required before the naive T cell can proliferate and differentiate into an armed effector cell, and that second signal is delivered by the interaction of CD28 molecules on the surface of the T lymphocyte with B7 molecules (B7-1 or CD80, and B7-2 or CD86) on the surface of an APC. Indeed, if the first signal (TCR:MHC-peptide) is delivered in the absence of the second signal, not only does the T cell fail to become an armed effector cell, it actually goes into an inactive state called anergy. It is believed that such anergic cells eventually undergo a slow process of programmed cell death called apoptosis. Because most cells in an animal or human do not express, nor can they be induced to express, B7 molecules, these normal cells cannot serve as APC. Therefore, when a T cell bearing a TCR specific for a self peptide interacts with a normal tissue cell bearing that self peptide expressed in a cell surface MHC molecule, the T cell is made anergic, and is unable to become an effector cell capable of killing the normal cell. The combination of central and peripheral tolerance eliminates most of the potentially autoreactive T cells that arise in the course of T cell development.

It is the APC (macrophages, dendritic cells, B cells) that have the ability to express large amounts of B7 molecules on their surfaces. However, it is important for the understanding of the present invention to recognize that, for example, the macrophage does not normally express large amounts of B7 on its surface. It has been demonstrated that surface expression of B7 is inducible, and when a macrophage interacts with certain microbial products (i.e., lipopolysaccharide or LPS), the gene coding for B7 is actively transcribed, and the macrophage begins to express B7 on its surface membrane. Only when the macrophage is activated in this way does it become an APC capable to providing both the first and second signal needed to cause specific T cells to proliferate and differentiate into armed effector cells.

To illustrate this further, let's take the example of the host immune response to an invading microorganism. The pathogen gains access to the blood or tissues of the host via one of several mechanisms, and begins to proliferate causing tissue damage. One of the first host defense cells to arrive on the scene is the macrophage, and the macrophage is capable of ingesting the microbes via the process of phagocytosis. Once the pathogen is ingested, it remains inside a vacuole that serves as a digestion chamber. Host cell enzymes are added to the chamber and the pathogen is killed and its proteins disrupted into peptides. The peptides are loaded onto newly formed MHC molecules, and then brought to the surface of the macrophage. The MHC:peptide complex is now ready to interact with a T cell bearing the correct TCR. At the same time, in order for the macrophage to be an effective APC, it must upregulate the expression of B7 genes, and begin to express large amounts of B7 costimulatory molecules on its surface. It is presently unclear just what microbial products are responsible for causing the upregulation of the B7 gene, and indeed, not all microorganisms ingested by macrophages cause the increased expression of B7 molecules. If sufficient B7 is expressed on the surface of the APC, the requisite T cells can be stimulated to become effector cells capable of killing the invading microorganisms.

It can be seen from this illustration of the function of the immune system that the B7 molecule and its surface membrane expression are key to the induction of the adaptive immune response. Failure of an APC to express B7 can actually result in the loss of protective T cells by the process of anergy. Should a T cell with an TCR specific for a peptide of an invading microorganism or tumor cell encounter that peptide on an APC that is not expressing B7, that T cell may be anergized and sent down the apoptotic pathway. This problem is addressed by the present invention.

We have used the expression “upregulate” herein, because the term has a well-defined meaning in this art. Although there is no specific verb form “to upregulate”, nor are its derivative forms upregulate, upregulation, upregulated, and upregulating found in the standard American Heritage Dictionary of the English Language, these terms have their root in the well understood verb form, to regulate. Moreover, the concept of upregulation is well understood by scientific practitioners as meaning an increase in the expression or amount of a particular substance. To demonstrate this usage, we performed a search of the National Center for Biotechnology Information's PubMed database for the inclusive years 1980 to the present, and found that the term upregulating was used in 715 biomedical research papers, the term upregulate was used in 2153 biomedical research papers, the term upregulates in 1799 papers, the term upregulated in 9872 papers and the term upregulation was used in 24,123 research papers. The first listed paper in the upregulation database series, Rohatgi et al., J. Neurosci. Res., 1980,73:246-254, used the terms upregulated and upregulation in the following sentence: “Levels of PAR mRNA for all four subtypes were upregulated as early as 6hr after unilateral ONC, except PAR-3, which showed a delayed upregulation.” In this article, the substances that increase in expression or amount are the mRNAs for protease-activated receptors (PARs). The final paper listed in this enormous database is an article by Takeuchi et al., Am. J. Physiol., 2003,238:G135-140, that used the term upregulation in the following manner: “The upregulation of the gastrin receptor was evident if the binding capacity was expressed per milligram of protein, per microgram of DNA, or per amount of 125I-labeled choleragen bound to the same membrane preparation”. In this paper, the substance that was increased in expression or amount was the receptor for the hormone gastrin. Although these papers were published 22 years apart, they used the term upregulation in the same way.

The first paper in the upregulating database series, Masri, Mol. Immunol. 2003,39:1073-1077, used the term upregulating in the following sentence: “Recently, antibodies to the CD40/CD40 ligand have been shown to induce long-term graft survival with the inhibition of the Th1 cytokines (INF), IL-2 and IL-12 and upregulating the Th2 cytokines IL-4 and IL-10”. Here, the term upregulating refers to increased expression of the TH2 cytokines IL-4 and IL-10. The final paper in this series, Fiorilli et al., Surv. Immunol. Res., 4 Suppl. 1:129-134, 1985, uses upregulating in the following manner: “The possibility of upregulating the immunoglobulins is of particular relevance in patients with hypogammaglobulinemias and this paper reports on the results of thymopentin treatment in 9 patients with selective IgA deficiency”. In this case, the substance that is increased in expression is immunoglobulin. Interestingly, one skilled in the art would immediately recognize thymopentin as the “upregulating agent” for immunoglobulin.

As used herein, the specific function of the upregulating agent is to increase the expression or amount of B7 family costimulatory genes and molecules in antigen presenting cells. Those skilled in this art will appreciate that we are referring to the increased expression of the B7 gene and the B7 molecule, and that the use of the term upregulate, by simple extension, recognizes the pharmacologic agent as an “upregulating agent”. A layman might choose to use the terms “increase” or “increasing” in place of “upregulate” or “upregulating” to confer the idea that there is more of a substance present as the result of the use of the agent. However, to an artisan skilled in the art of molecular biology, the terms upregulate and upregulating have clear meaning.

We sought to identify a safe and effective pharmacologic agent that could upregulate the expression of the critical B7 costimulatory molecule on APC. Such an agent can be administered to an animal or human in a dosage sufficient to cause the cell surface expression of B7 on macrophages (or other APC), thereby allowing the macrophage to better serve the function of antigen presentation to T lymphocytes. Macrophages have many receptors for highly conserved microbial constituents, and the interaction of the ligand on the microbe with its receptor on the macrophage has been shown, for example in the case of the LPS receptor, to cause the activation of the macrophage. Macrophages also have glucan receptors, receptors into which β1,3-glucan-containing ligands will bind. There is considerable evidence that the β1,3-glucans cause the activation of macrophages, making them more effective at phagocytosis and killing of microorganisms. The β1,3-glucans have been administered to animals and humans for years with no untoward effects, so we wondered whether this class of pharmacologic agents would upregulate the cell surface expression of B7 molecules on macrophages. We performed a laboratory experiment in which macrophages taken from the peritoneal cavity of donor mice, or tumor macrophages were incubated in vitro with various amounts of β1,3-glucan-containing compositions (see below). By using fluorescent-labeled anti-B7 antibodies, we discovered that whereas both the peritoneal macrophages and the tumor macrophages did not express large amounts of cell surface B7 molecules before the addition of P 1,3-glucan-containing compositions, after incubation with the β1,3-glucan-containing composition these cells began to express large amounts of B7 molecules on their surface membranes. Our invention, therefore, involves the identification of β1,3-glucan-containing compositions as pharmacologic agents capable of causing macrophages to express surface membrane B7 molecules. Our invention includes the administration of such β1,3-glucan-containing compositions to animals and humans to increase the effectiveness of APC in providing the critical second signal necessary for inducing specific T lymphocytes to proliferate and differentiate into armed effector cells capable of protecting against harmful non-self, like pathogenic microorganisms and cancer.

Although the B7 gene has been cloned and methods described for its expression in various cell types, we do not need to clone the B7 gene and manipulate its expression in a host cell in order to augment an immune response.

This invention relates, generally, to a method of augmenting the immune response to a foreign antigen (i.e., pathogenic microorganism, tumor) by the use of β1,3-glucan-containing compositions. The invention involves the administration of the β1,3-glucan-containing composition to an animal or human in a dose and by a route that serves to bring a critical amount of this material to the vicinity of macrophages or monocytes (blood, tissues, secondary lymphoid organs). The β1,3-glucan-containing composition interacts with a specific glucan receptor on the surface of these APC, initiating an intracellular signal that results in the upregulation and surface expression of a family of molecules called B7 (i.e., B7. 1 and B7.2). The B7 molecules are costimulatory molecules that are critical in the provision of a second signal to specific T lymphocytes that have received a first signal through a specific cell surface receptor (TCR) that interacts with a foreign peptide in the context of an MHC molecule on the surface of the APC. When the naive T lymphocyte has received both signals, it proliferates and then differentiates into an armed effector T lymphocyte that can effectuate one of the important defense mechanisms of the immune response (i.e., cell mediated immunity or antibody-mediated immunity). Because APC like macrophages do not normally express much cell surface B7, they are not effective in delivering the all critical second signal to T lymphocytes. Our discovery that β1,3-glucan-containing compositions can cause APC like macrophages to express large amounts of B7 on their surfaces, allows us to provide a pharmacological intervention to make the APC more effective in initiating the adaptive immune response, and therefore, more effective in providing protection against foreign antigens like microbes and tumors.

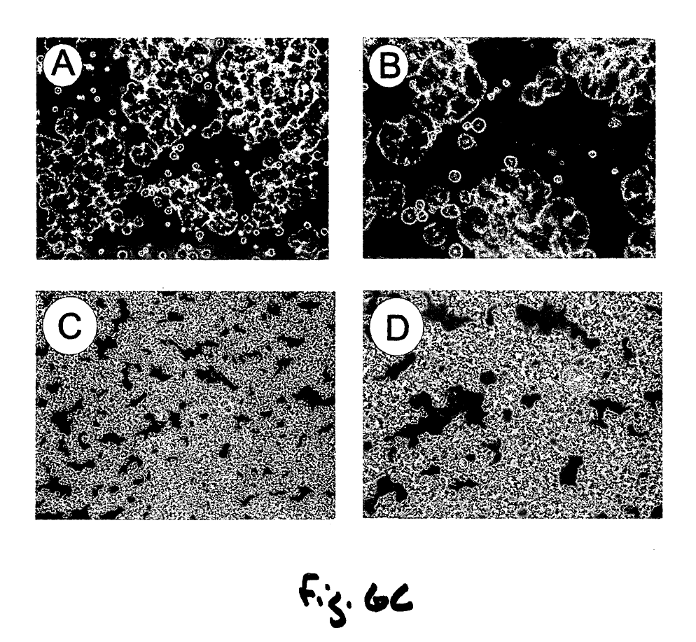

Referring now to FIGS. 6-8 of the drawings and to the Examples 5,6,7,8 and 9, there is a clear teaching that in accordance with the present invention, a β1,3-glucan-containing composition causes the upregulation of cell surface expression of B7 molecules on microphages. We also demonstrate that the same β 1,3-glucan-containing composition can activate macrophages and enhance their rate of bacterial phagocytosis. We further demonstrate the enhancement of an immune response to a foreign antigen (sheep red blood cells) in mice given the β 1,3-glucan-containing composition orally.

For these experiments we used a preparation of β 1,3-glucan from the common Baker's yeast Saccharomyces cerevisiae, but another source of β 1,3-glucan could easily be derived from other yeasts, bacteria, and plants. We used a common method of preparing an alkali extract of the yeast, which was then subjected to sonication in order to disrupt the larger glucan globules, preferably, into smaller particles in the size range of 1-2 microns. However, our invention is meant to encompass any β 1,3-glucan-containing composition, ranging from whole Baker's yeast to purified soluble β 1,3,-glucan, that upon interaction with a macrophage causes the upregulation and cell surface expression of B7 molecules.

### EXAMPLE 5

Mouse Tumor Macrophage Cell Line (P388D1) Treated in vitro with a β 1,3-glucan-containing Composition: Upregulation of B7 Surface Membrane Expression.

In this experiment the tumor macrophage-like cell line P388D1 (ATCC, Manassas, Va.) was grown in wells of eight-chambered microscope slides containing tissue culture media with 10% fetal bovine serum at 37° C. in 5% CO₂. Cells were stimulated with media (control cells) or media containing 100 μg/ml β 1,3-glucan-containing composition for 1 hour. During this time, it was demonstrated (data not shown) that the macrophages ingested large quantities of the β 1,3-glucan-containing particles. After incubation, the stimulant was washed away and replaced with growth media and incubated as before. Approximately 24 hours post-stimulation, live cells were incubated with a fluoresceinated antibody directed to mouse B7.2 (BD PharMingen, San Diego, Calif.). After an incubation of 1 hour, the unbound antibody was washed away and the cells were examined under a Nikon Eclipse 400 fluorescence microscope. Upon examination of more than 50 cells, it was concluded that the glucan treatment had the effect of increasing the amount of surface membrane B7.2 from negligible before (FIG. 6A) to substantial after treatment (FIG. 6B). Note the bright yellow halo around the macrophages in the glucan-treated cells. From this experiment, it is concluded that a β 1,3-glucan-containing composition can cause the upregulation of a B7 costimulatory molecule in this particular mouse macrophage-like tumor cell line.

### EXAMPLE 6

Mouse Peritoneal Macrophages Treated in vitro with a β 1,3-glucan-containing Composition: Upregulation of B7 Surface Membrane Expression.

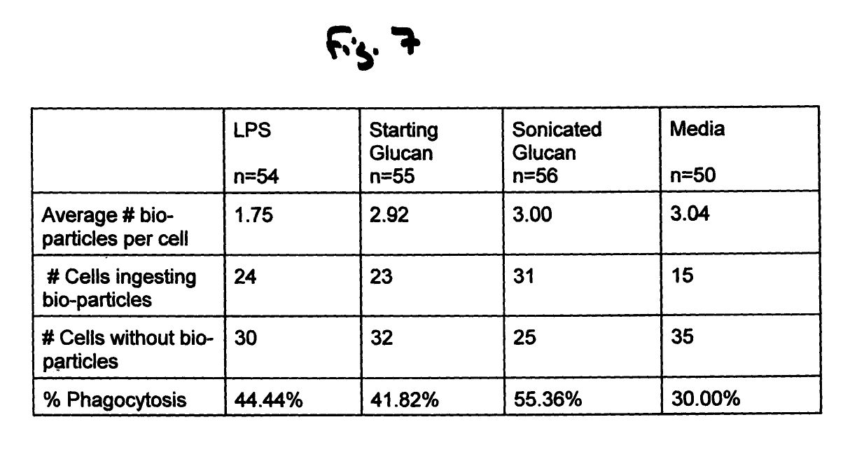

In this example of the upregulation and surface expression of the B7.2 costimulatory molecule on mouse macrophages by a β 1,3-glucan-containing composition, mouse peritoneal macrophagse were harvested by peritoneal lavage with cold tissue culture medium. The cells were placed in tissue culture medium containing 10% fetal bovine serum and placed in the wells of eight-chambered microscope slides and incubated at 37° C. and 5% CO₂ for two hours. The non-adherent cells were removed by washing in warm medium, and the macrophages were found to attach to the glass substrate of the microscope slide. Cells were stimulated with media (control cells) or media containing 100 μg/ml β 1,3-glucan-containing composition for 1 hour. During this time, it was demonstrated (data not shown) that the macrophages ingested large quantities of the β 1,3-glucan-containing particles. After incubation, the stimulant was washed away and replaced with growth media and incubated as before. Approximately 24 hours post-stimulation, live cells were incubated with a phycoerythrin-conjugated antibody directed to mouse B7.2. After an incubation of 1 hour, the unbound antibody was washed away and the cells were examined under a Nikon Eclipse 400 fluorescence microscope. Upon examination of more than 50 cells, it was concluded that the glucan treatment had the effect of increasing the amount of surface membrane B7.2 from negligible before (FIG. 7A) to substantial after treatment (FIG. 7B). Once again, note the bright yellow hallo around the glucan-treated cells. From this experiment, it is concluded that a β 1,3-glucan-containing composition can cause the upregulation of the B7 costimulatory molecule on mouse peritoneal macrophages in vitro.

### EXAMPLE 7

Mouse Peritoneal Macrophages Treated in vivo with a β1,3-glucan-containing Composition: Upregulation of B7 Surface Membrane Expression.

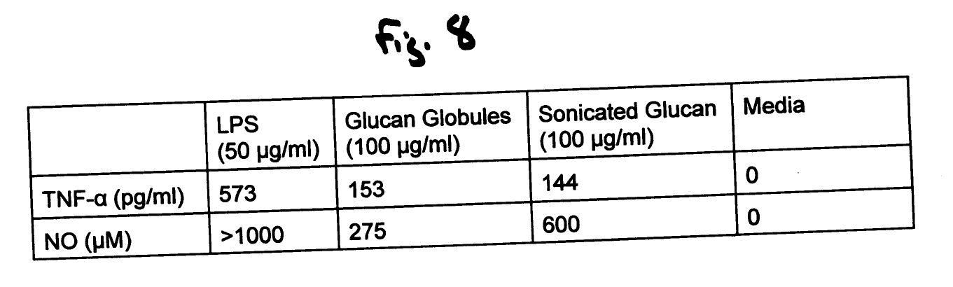

In this experiment, mice were injected via the intraperitoneal route with 100 μg of a β 1,3-glucan-containing composition in 0.25 ml of sterile saline or with 0.25 ml sterile saline alone. After 24 hours, peritoneal macrophages were harvested and processed for immunofluorescence as described in Example 6 using a phycoerythrin-conjugated antibody toward mouse B7.2. Upon examination of more than 50 cells, it was concluded that the glucan treatment had the effect of increasing the amount of surface membrane B7.2 from negligible before (FIG. 8A) to substantial after treatment (FIG. 8B). From this experiment, it is concluded that a β 1,3-glucan-containing composition administered to a mouse by the intraperitoneal route can cause the upregulation of the B7 costimulatory molecule on mouse peritoneal macrophages.

### EXAMPLE 8

Mouse Peritoneal Macrophages Stimulated in vivo with a β 1,3-glucan-containing Composition: Enhanced Phagocytosis of Bacteria.

In this experiment mice were fed 132 μg/kg of a β 1,3-glucan-containing composition daily for 18 days. Peritoneal macrophages were harvested by peritoneal lavage as described in Example 6. Peritoneal macrophages were plated into each well of a 8-well chambered slide. Macrophages were allowed to adhere for approximately 4 hours followed by washing 3× with PBS to remove non-adherent cells. 100 μl of fluoroscein isothiocyanate-labeled bacteria (bioparticles, 1:20 dilution in PBS) was added to each well and incubated for 20 minutes at 37 ° C. Following incubation, the slides were rinsed in PBS to remove excess bio-particles. Bio-particles remaining on the outside of the macrophages were quenched by the addition of 100 μl of a 50 μg/ml ethidium bromide solution to each well and incubated for 10 minutes at room temperature in the dark and then analyzed by fluorescence microscopy. The results are shown in Table 1. It was concluded from this experiment that a β 1,3-glucan-containing composition can activate macrophages for the enhanced engulfment of microorganisms.

TABLE 1

| | | | Ave# |
|---|---|---|---|
| | Cells w/o | Cells with | particles/ |
| | particles | particles | cell |
| No Glucan | 31.8 (64.9%) | 17.2 (35.1%) | 2.2 |
| Glucan | 18.7 (36.0%) | 33.3 (64.0%) | 3.4 |

### EXAMPLE 9

Mouse Peritoneal Macrophages Stimulated in vivo with a β 1,3-glucan-containing composition: potentiation of immune responses.

This experiment was done in two parts. Mice were fed 132 μg/kg of a β 1,3-glucan-containing composition daily for 19 days. On day 2 and day 8, mice were given an injection of 1×10 sheep red blood cells (SRBC) via the intraperitonel route. For the detection of serum anti-SRBC antibodies, blood was drawn from the mice eleven days after the last injection and allowed to coagulate. After centrifugation the serum component was aspirated and used for hemagglutination assays. Serum was serially diluted from 1: to 1:20,480 in PBS in a 96-well microtiter plate. An equal amount of 1.5% washed SRBC was added to each serum dilution, mixed well and incubated at 4° C. for 6 hours. The reciprocal of the final dilution causing noticeable agglutination of the SRBC was defined as the titer. The results are shown in Table 2.

TABLE 2

| | Reciprocal |
|---|---|
| Treatment | Titer |
| No glucan | 1841.1 |
| Glucan | 2906.7 |

It was concluded from this experiment that the administration of oral glucan potentiates the primary IgM antibody response to a T cell dependent antigen (SRBC).

In the second part of the experiment mice, spleens were harvested aseptically from the same mice eleven days after the last injection of SRBC. The tissue was dissociated by mincing followed by sieving through a 210 μm polypropylene mesh into growth medium. After lysis of red blood cells the lymphocytes were counted and diluted to 1×10⁸ cells/ml for plaque-forming cell assays to determine the number of IgM secreting B cells. A 1:100 dilution of spleen cells was mixed with a 7.5% SRBC solution, placed in a Cunningham-Szenberg chamber and incubated at 37° C. for two hours. After incubation the plaques were enumerated. The results are shown in Table 3.

TABLE 3

| | |
|---|---|
| 0 mg Glucan | 175 ± 50 |
| 10 mg Glucan | 625 ± 275 |

It was concluded from this experiment that the administration of oral glucan increases the number of B lymphocytes making antibodies to SRBC, another example of the immunopotentiating ability of β 1,3 glucan.

The preceding examples are not intended limit the scope of Applicants' invention, but serve as an explanatory tool in the many advantages of Applicants' invention. Further, although various embodiments of Applicants' invention have been described in the preceding description, it will be understood by those skilled in the art that various embodiments fit within the scope of the invention.

The present invention also relates generally to a novel composition of small particle size beta glucan which contains partially deacetylated N-acetylglucosamine that provides a free amino group for vaccine conjugation and also provides stability to the microparticles, particularly during transit through the gastrointestinal system. The invention also pertains to the method of manufacture of the small particle size beta glucan which contains partially deacetylated N-acetylglucosamine and the use of this beta glucan for vaccine conjugation and in the administration of vaccine.

Perhaps the most significant contribution to human and veterinary medicine over the last century has been the process of vaccination. However, infectious diseases still represent the most important causes of morbidity and mortality worldwide, in part because there are numerous infectious diseases affecting billions of humans, for which even moderately efficacious vaccines are not available (e.g., malaria, schistosomiasis, AIDS). Moreover, notwithstanding the incredible successes of certain vaccines (e.g., Vaccinia for smallpox), many present vaccines fail to induce the levels of protective immunity in humans that have been achieved in animals. One reason for this mediocre vaccine performance is the lack of a suitable vaccine adjuvant for human use. Broadly defined, an adjuvant is a substance that can augment the immune response to vaccine antigens (Cox and Coulter, 1997). Adjuvants have been around since 1926 when Ramon and his colleagues (Ramon, 1926) discovered that horses given intradermal immunizations of diphtheria toxoid made higher antibody responses if they developed bacteria-laden abscesses at the injection site. However, even today we do not understand the mechanism of action of most adjuvants. Indeed, in 1989 the noted immunologist Charles Janeway (Janeway 1989) referred to adjuvants as “the immunologist's dirty little secret.” Even the prototypical complete Freund's adjuvant (CFA), a combination of mineral oil and mycobacteria (Freund, Casals, and Hosmer, 1937), is incompletely understood mechanistically, though it strongly enhances immune responses to most antigens. There is evidence, however, that components of the mycobacteria, especially muramyl dipeptide, up-regulate the expression of a variety of cytokines and importantly, co-stimulatory molecules. Unfortunately, CFA causes such a profound inflammatory response that it cannot typically be used in humans. On the other hand, many so-called adjuvants simply provide an inert matrix to which the vaccine antigens are attached, and while they may promote the persistence and endocytosis of vaccine antigens, do not induce to a significant extent co-stimulatory molecules on APCs.

There is a long-felt need for additional vaccine adjuvants that are safe yet effective for use in humans and other animals, and this has become even more pressing with the recent threat of bioterrorism. Our increased understanding of the intricacies of the immune response has allowed the rational design of new vaccines. Some of the risks associated with crude killed or live attenuated vaccines have been mitigated through the engineering of highly purified component vaccines. Indeed, we now have access to so-called subunit vaccines comprised of small, chemically defined components of microorganisms. Ironically, as some of our newer vaccines have become more “pure,” they have also become less immunogenic. One explanation for this phenomenon is the inability of highly purified component vaccines to appear “foreign” or to elicit so-called “danger signals”, thereby failing to initiate the adaptive immune response. For protein antigens to effectively elicit an adaptive immune response, they must not only be processed and presented by APCs to T cells, but the APCs must also express so-called co-stimulatory molecules.

Co-stimulation is one of the most ignored concepts in vaccine design today. The induction of an immune response to most carbohydrate and all protein antigens requires both antigen recognition by cognate receptors on immune cells (e.g., T cell receptors), and the simultaneous interaction of co-stimulatory ligands on APCs (e.g., B7 family glycoproteins) with their counterpart receptors (CD28) on T cells. It is also important to note that failure of a vaccine antigen preparation to induce the expression of co-stimulatory molecules can lead to the phenomenon of tolerance. If a vaccine induces tolerance, not only does it fail to initiate an adaptive immune response, but the specific T cells that interact with the vaccine peptides presented in the context of MHC molecules on APCs become anergic and ultimately undergo apoptosis. Therefore, a rational approach to enhancement of vaccination is to use an adjuvant that induces the concomitant expression of co-stimulatory molecules and/or to design antigens that induce co-stimulation directly, or to add to the vaccines and antigenic substances that induce the expression of co-stimulatory molecules.

As mentioned before, the B7 family molecules are known to be potent co-stimulatory ligands that interact with CD28 receptors on T cells to generate the so-called second signal that complements the T cell receptor:MHC:peptide first signal. The combined antigen-specific first signal and co-stimulatory second signal facilitates the proliferation and differentiation of T cells into armed effector cells. Since expression of co-stimulatory molecules on macrophages and dendritic cells bearing MHC:peptides allows for successful antigen presentation to T cells and initiation of the adaptive immune response, a combination of vaccine antigens and our microparticulate beta-glucan would strongly enhance the adaptive immune response. Indeed, one of the most critical attributes of an adjuvant is the ability to induce the expression of co-stimulatory molecules, particularly for vaccine antigens that do not inherently induce co-stimulation.

In general, glucan preparations evaluated for immune enhancement can be divided into soluble and particulate, and both forms have demonstrated immune enhancing properties. Our procedure for making microparticulate beta-glucan preserves chitin, while eliminating most of the contaminating mannan and protein. The preservation of chitin, as explained below, is significant.

The present invention relates to a novel microparticulate beta-glucan that can be used as a vaccine adjuvant, to methods of manufacturing this new microparticulate beta-glucan, to conjugates of this microparticulate beta-glucan and vaccine antigens, and to pharmaceutical formulations of these conjugates useful as vaccine adjuvants in animals and humans. This new microparticulate beta-glucan binds to glucan receptors on a variety of phagocytic cells and enhances their immunological functions (e.g., cytokine production), and contains around 1-10% deacetylated N-acetylglucosamine, preferably about 4%, that provides a free amino group for vaccine conjugation and also provides stability to the microparticles, particularly during transit through the gastrointestinal system, and has particles which are predominantly 0.3-3 microns in size and often closer to 1-2 micron particles that are ideally suited to phagocytosis by macrophages and dendritic cells, and causes the expression of co-stimulatory molecules on antigen presenting cells (APCs), a critical requirement for inducing an adaptive immune response. The microparticulate glucan of the present invention enhances the immune response to vaccine antigens, and thus serves as a safe and effective vaccine adjuvant.

As the glucan re-aggregates, some of the beneficial effect of the glucan is not achieved because the macrophage receptors are not activated as readily by large particle size or reaggregated glucan. As the glucan re-aggregates, it appears to pass through an animal or human digestive system without substantially complete absorption. There has been a long-felt need for microparticle beta-glucans which retain a small percentage of chitin in the form of partially deacetylated N-acetylglucosamine and which do not reaggregate but rather remain substantially in a particle size of about 0.3-3, preferably, 1-2 microns in diameter throughout preparation, packaging, and/or use without re-aggregation.

Further, the art field has the unmet need for reliable and more effective vaccine adjuvants which are complimentary and/or enhance immunological responses and processess and immunity.

The process set forth below describes a novel method for preparing a microparticulate glucan that can be used as a vaccine adjuvant. The first aspect of this invention involves the process of preparing the microparticulate glucan from a source of glucan. The process of the present invention uses a less rigorous combination of alkali and acid extractions of the starting yeast material in order to preserve around 1-10% chitin preferably containing at least 4% chitin, the usefulness of which will be apparent later in this description. Furthermore, the present invention creates particles which are predominately 0.3-3 microns in size, and preferably 1-2 microns in size that have a variety of advantages over larger glucan particles.

It should be noted that while S. cerevisiae is a convenient source of glucan, other sources of beta-glucan may be substituted as long as these sources have chitin as a constituent of their cell walls, or alternatively, chitin or chitin containing compounds can be added to the beta glucan or glucan containing compounds. Chitin is a polymer which naturally occurs and is found in a variety of cells in yeast, fungi, mushrooms, and may also be obtained from mutant or genetically altered cells.

Generally any type of yeast can be used, including mutant or genetically altered yeasts such as Saccharomyces cerevisiae, Saccharomyces delbrueckii, Saccharomyces rosei, Saccaromyces microellipsodes, Saccharomyces carlsbergensis, Saccharomyces bisporus, Saccharomyces fermentati, Saccharomyces rouxii, Schizosaccharomyces pombe, Schizophyllum commune, Scierotium glucanium, Lentinus edodes, Kluyveromyces lactis, Kluyveromyces fragilis, Kluyveromyces polysporus, Candida albicans, Candida cloacae, Candidatropicalis, Candida utilis, Hansenula wingei, Hansenula arni, Hansenula henricii, Hansenula americana, Hansenula canadiensis, Hansenula capsulata, Hansenula polymorpha, Pichia kluyveri, Pichia pastoris, Pichia polymorpha, Pichia rhodanensis, Pichia ohmeri, Torulopsis bovina and Torulopsis glabrata. If yeast is used as the source of glucan, it should also be noted that the yield of microparticles is increased substantially if the yeast is grown to late log phase to increase the maximize budding, and therefore, the number of bud scars per yeast cell. The initial process comprises the steps of: (a) extracting the active dry yeast one time with an alkali solution to remove alkali-soluble material; (b) extracting the alkali-insoluble material one time with acid to remove acid soluble material; c) extracting acid insoluble material from step (b) with an organic reagent to remove residual lipid. The active dry yeast is added to an alkali solution such as 2-6% NaOH or other base commonly known or used in the art with stirring such as by automation about 30-60 minutes or thereabout. The material is then heated to 115° C.-145 at about 8.5-12 psi for about 45-75 minutes and allowed to settle for 72-96 hours. Also, unlike most other glucan preparation procedures described in the prior art, this alkali extraction is generally performed only once. The sediment is resuspended and washed in dH₂O by centrifugation (e.g., 350 ×g for 20 minutes). The alkali insoluble solids are combined with an acid such as 4%-6 acetic acid or other acid commonly known or used in the art and heated to 85° C.-100 for about 1-4 hours, then allowed to settle at 38-42° C. The acid insoluble solids are drawn off and centrifuged as above. This acid extraction is generally performed only once. The compacted solid material is mixed with a peroxidating agent such as 3% H₂O₂ and refrigerated for 3-6 hours with periodic mixing. The material is then centrifuged and the pellet washed twice with dH₂O followed by two washes in 100% acetone or other organic solvent. The harvested solid material is dispersed on drying trays and dried under vacuum at 38-42° C. for 2-4 hours in the presence of Ca₂SO₄, and then further dried overnight under vacuum at room temperature. This procedure yields a white powder with about 2-5% protein and lipid. Carbohydrate analysis reveals about 85-95% hexoses (glucose) with between 1-10% chitin (measured as N-acetylglucosamine). It may also be possible to use weaker acids and bases to extract the beta glucan and chitin containing polysaccharide compounds, and extract the water insoluble residues multiple times, while obtaining a more purified beta glucan which still retains the desired chitin in the form of partially deacetylated N-acetylglucosamine.

Examination of a saline suspension of the resultant N-acetylglucosamine and glucan-containing powder from the process described above exists predominantly in the aggregated form, typically ranging from 5-100 microns in diameter. As noted previously, it is desirable to reduce the aggregate size to predominantly about 0.3-3 micrometers, and preferably predominately 1-2 microns in diameter.

To make a predominately 0.3-3 micron diameter microparticulate preparation that does not substantially reaggregate upon drying and rehydration, the aggregated glucan is first hydrated in dH₂O overnight at room temperature, and then a 1.5% suspension of the hydrated material is subjected to sonic energy via a 19 mm probe utilizing a 300 V/T Sonic Dismembrator (BioLogics, Gainesville, Va.). Using an ultrasonic output frequency of 20 kilocycles per second at 192 watts, the glucan suspensions are sonicated on ice for 12 minutes(for example, twelve 48 second cycles of sonication with a 12 second pause between cycles). Experimental studies have shown that excessive sonication of the glucan creates heat that may denature the glucan and shortened life of the sonic probe. Accordingly, care should be taken not to over sonicate the glucan and to provide a sufficient time in between cycles to allow the probe to cool. The process of the most preferred embodiment will dissociate substantially all of the glucan aggregates to particles of about 0.3-3 microns in diameter, preferably 1-2 microns.

The sonicated glucan suspension is spray-dried using a spray dryer such Buchi 190 Mini-Spray Dryer (Buchi, Germany), or other such spray dryer or other spray drying process known or used by one skilled in the art. The sonicated suspension is preferably spray dried by such a dryer with an inlet air temperature of 110-170° C., and outlet air temperature of 90-120° C., and an atomizer pressure of 30-100 psi. Using flow cytometric analysis with an EPICS XL-MCL Flow Cytometer (Coulter Electronics, Hialeah, Fla.), 1 mg of sonicated glucan consists of approximately 1.81×10¹¹ microparticles in the size range of 0.3-3 microns. However, other settings and spray dryers may be used and be within the scope of the present invention varying the quality of the sprayed glucan. In fact, other settings may be required when using a different spray dryer. The preferred spray dryer produces a finely sprayed glucan powder that does not re-aggregate into glucan globules. A fine, non-aggregated powder, is formed from the spraying. The resulting glucan containing composition, existing substantially as a powder, may then be loaded into capsules, pills or other containers. The powder may also be stored and later re-hydrated for future use. When this dried material is rehydrated in an aqueous medium, it remains substantially unaggregated.

The cell walls of S. cerevisiae are composed of four major components beta-1,3-(D)-glucan (ca. 40%); beta-1,6-(D)-glucan (ca. 14%); mannoprotein (ca. 41%); and N-acetylglucosamine or chitin (ca. 5%). Our new process utilizes less rigorous alkali and acid extractions in order to preserve most of the chitin. Indeed, the particle size and chemical composition of our new microparticulate beta-glucan preparation is similar to that described for yeast bud scars. The chitin in our formulation also provides needed chemical and mechanical stability to the microparticles. The microparticulate beta-glucan of the present invention also survives transit through the stomach, duodenum, and jejunum, and can be found intact in the ileum associated with M cells overlaying the Peyer's patch lymphoid tissue. Because the microparticulate beta-glucan of the present invention has between 1-10% chitin that has been partially deacetylated by the alkaline treatment and bears a reactive primary NH₂ group, a variety of chemical procedures can be used to attach any type of vaccine or vaccine antigens and other type of non-traditional molecules and components to the particles such as polypeptides for gene therapy, genes or genes involved in the treatment of cancer or tumors, and nucleic acids for gene expression. The vaccines can also be administered to the subject human or other animal through any route.

One such procedure involves water soluble carbodiimide. For vaccine antigens that cannot be attached to the NH₂ functionality, they can be directly attached to the glucan by a variety of chemical reactions, such as via a maleimide linkage group. Since our microparticulate beta-glucan preparation has only trace amounts of protein, we can determine the amount of vaccine protein attached using the micro-Bradford protein assay. Because the number of vaccine antigen molecules per particle may profoundly influence the immunization success, reaction conditions can be varied to prepare conjugates with various amounts of attached vaccine protein. To more precisely determine the reaction conditions needed to attach a specific number of vaccine antigen molecules per particle, vaccine antigens can be tritium-labeled and standard radioactive binding analyses can be performed.

Again, this water insoluble beta-glucan preparation which provides a free amino group for conjugation and which can be used as a vaccine adjuvant, comprises microparticulate beta-(1,3)-glucan with or without beta-(1,6)-glucan side chains which do not substantially reaggregate upon drying or rehydration, at least 4% by weight partially deacetylated N-acetylglucosamine within the beta-glucan that provides a free amino group for vaccine conjugation. A vaccine or an antigenic substance is then conjugated with the free amino group on the adjuvant. The adjuvant with or without the conjugated vaccine or antigenic substance may be in an aqueous suspension or solid such as a tablet, or in a colloidal mixture. The source of this glucan may be obtained from a yeast cell wall extract, or a fungus or mold cell extract. The adjuvant preferably contains less than or about 3%-5% by weight protein and lipid, and more than or about 85%-95% by weight glucose, and about 1%-10% by weight chitin or partially deacetylated N-acetylglucosamine, but could also contain more or less of these constituents.

Sugars, such as maltodextrin may also be added as a filler, and gelatin may be added as a filler. Other fillers used or known by one skilled in the art may also be used.

### EXAMPLE 10

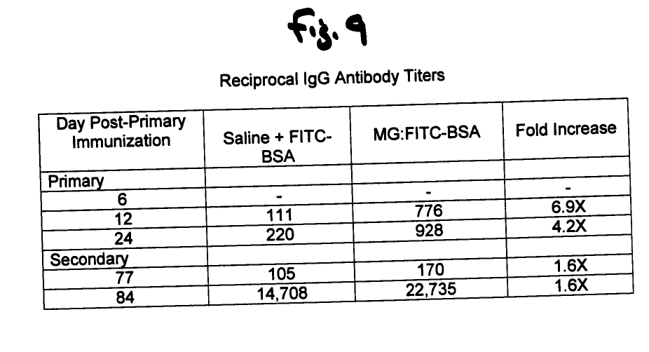

In FIG. 9 of the drawing there is provided an illustration of tabular results of a vaccination study in which a prototypic vaccine antigen (fluorescein isothiocyanate-labeled bovine serum albumin, or FITC-BSA) is administered intradermally to BALB/c mice in either normal saline or as a conjugate with microparticulate glucan (MG). In both cases, the amount of vaccine material administered to each of five mice was 40 μg. Serum was collected from each of the five mice per group on days 6, 12, and 24 after the primary immunization. These sera were tested by enzyme-linked immunosorbent assay (ELISA) to determine the average IgG anti-FITC-BSA antibody titers, and the data shown in the table represent the reciprocal of the IgG anti-FITC-BSA titers (e.g., a 1/1000 titer is designated 1000). It can be seen from the table that at days 12 and 24 post-primary immunization, the mice given FITC-BSA vaccine in saline has antibody titers of 111 and 220 respectively, whereas the mice given the same amount of vaccine conjugated to MG had titers of 776 and 928 for days 12 and 24. On days 12 and 24, the antibody titers were 6.9 times and 4.2 times higher in the MG-FITC-BSA group than in the saline FITC-BSA control group.

On day 70, both groups of mice were reimmunized with the same vaccines as given for the primary immunization, and their sera sampled on days 77 and 84. As can be seen in the table, the IgG antibody titers to FITC-BSA had diminished, but after immunization, the MG-FITC-BSA group showed average antibody titers 1.6-fold higher than the saline control group. By day 84, the saline control group showed a reciprocal titer of 14,708, while the MG:FITC-BSA group had average reciprocal titers of 22,735. The increase seen in the MG:FITC-BSA groups was 1.6-fold higher than in the saline control group.

These data showed that MG can augment both the primary and secondary IgG antibody responses to a vaccine (FITC-BSA), thus demonstrating that MG can serve as a vaccine adjuvant.

There are many ways in which an adjuvant can influence the immune response to a vaccine antigen (e.g., activation of APCs for enhanced phagocytosis, upregulation of MHC expression, improved antigen processing). However, while these are important, unless the APC is induced to express co-stimulatory molecules, an adaptive immune response will not occur. We have determined that microparticulate beta-(1,3)-glucan is a strong inducer of the expression of the B7 co-stimulatory molecules, and thus should promote the presentation of vaccine antigen to T cells. Accordingly, a method of using microparticulate beta-(1,3)-glucan as a vaccine adjuvant is also contemplated which generally comprises the steps of preparing or obtaining a microparticulate beta-(1,3)-glucan composition which does not substantially reaggregate upon drying and rehydration which contains partially deacetylated N-acetlyglucosamine with a free amino group, suspending or maintaining the microparticulate beta-(1,3)-glucan composition in liquid, adding at least one vaccine or antigenic substance, conjugating the vaccine onto the free amino group, and administering the vaccine to an animal or human. This method also contemplates that the glucan will be composed of a yeast cell wall extract and/or algae, and/or fungi, but there may also be other such suitable sources used or known by one skilled in the art.

The vaccine adjuvant may also be stored after preparation and before the vaccine or antigenic substance is added, or conversely stored after the vaccine or antigenic substance is added. There are many methods available and known to one skilled in the art for conjugating a vaccine to particle such as the novel beta-glucan particle of this invention. Further, depending upon the route of administration, the microparticulate composition may be sterile or sterilized before it is used and/or containerized and stored. Similarly, if the liquid, colloidal, or powder form of the adjuvant is containerized, liquid and/or vaccine and/or antigenic substances may be added to the container before administration. In this method, the adjuvant preferably contains less than or about 2%-5% by weight protein and lipid, and more than or about 85%-95% by weight glucose, and about 1%-10% by weight chitin or partially deacetylated N-acetylglucosamine, but could also contain more or less of these constituents. In this method, sugars or salts which are used or known by one in the art may added. The resultant vaccine may be administered by any route, including but not limited to intradermally or subcutaneously, intramuscularly, intraperitoneally, orally, nasally, externally, or into the lymphatic system or blood stream. If the vaccine is given intramuscularly, a depot effect may occur after vaccination.

In this method, the conjugated vaccine may also provide protection from protease degradation. The conjugated vaccine may also target antigens to Peyer's patches for processing and presentation to T-cells. Furthermore, this method contemplates that if orally adminstered, oral tolerance may be minimized or eliminated.

As stated, there are many advantages of this adjuvant such as enhancing he immunological effects of a vaccine or antigenic substance. The immunological effect may be measured or quantified in terms of a serum antibody unit, which may be enhanced or increased. Further the immunological effects or response of the vaccine occur sooner in the terms of a serum antibody unit. Further the enhancement of immunological effects, wherein the serum antibody unit is measured by hemagglutination inhibition (HAI) or passive hemagglutination (PHA), and may also include the action of enhancing the immunological effects is an action of promoting IgA. In this conjugated form, co-stimulation and proliferation of antigen-specific T-cells may occur.

The adjuvant may be in a dry form, in an aqueous solution, or colloidal suspension. The adjuvant may also be containerized and stored until use. The glucan in the adjuvant may be obtained from a yeast cell wall extract, fungus or mold cell extract, or other such suitable sources known or used by one skilled in the art. Further, the glucan in the adjuvant may contain less than about 2-5% by weight protein and lipid, about 85-95% by weight glucose, and about 1%-10% by weight chitin or partially deacetylated N-acetylglucosamine. Other compositions may also be suitable as long as it contains an adequate amount of chitin for conjugation. Further, the adjuvant may contain fillers such as sugar, particularly, maltodextrin, and/or gelatin. The use of other suitable fillers known or used by one skilled in the art are also contemplated by this invention. Further, the use of preservatives or anti-oxidants may be appropriate depending upon the type of vaccine or antigenic substance used and depending upon whether the adjuvant and/or conjugated vaccines or antigenic substances are stored. The adjuvant may also be premixed or incorporated into food or drink, and may have added oils, lipids, preservatives and/or antioxidants, and organic solvents or inorganic solvents known or used by one skilled in the art, as necessary or desired.

Further, this invention also includes the conjugates of vaccines and other antigenic substances that are attached to the free amino group of microparticulate beta-(1,3)-glucan and which stabilizes the vaccine(s) and other antigenic substances and enhances the immunologic effects of vaccine. The conjugates are comprised of microparticulate beta-(1,3)-glucan with or without beta-(1,6)-glucan side chains and partially deacetylated N-acetylglucosamine within the beta-glucan that provides a free amino group for conjugation with the vaccines and other antigenic substances and which does not substantially reaggregate upon drying or rehydration and a vaccine or vaccines or an antigenic substance, wherein the vaccine or antigenic substance is conjugated with the free amino group. Again, the microparticulate beta-glucan has particles which are predominantly 0.3-3 microns in size, preferably 1-2 microns. Further this beta glucan contains about 2-5% protein and lipid and carbohydrate analysis reveals about 85-95% hexoses (glucose) with between 1-10% chitin (measured as N-acetylglucosamine).

The conjugated vaccine or antigenic substance may be administered once, or multiple times. Further, the conjugation of the vaccine or antigenic substance may be enhanced in terms of a serum antibody unit, hemagglutination inhibition (HAI), or passive hemagglutination (PHA).

What is claimed is:

1. An improved method for enhancing immune responses by upregulating co-stimulatory molecules, the upregulating of the co-stimulatory molecules comprising the steps of administering a glucan-containing composition to an animal or a human, in sufficient dosage to cause an enhanced expression of co-stimulatory molecules on antigen presenting cells, the co-stimulatory molecules providing a second signal to T lymphocytes, causing the T lymphocytes to differentiate into armed effector cells.

2. The improved method of claim 1 wherein the glucan-containing composition is at minimum a portion of a glucan selected from the group consisting of β 1,3-glucans and β 1,6-glucans.

3. The improved method of claim 1 wherein the molecule expressed is a molecule from a family of B7 molecules.

4. The improved method of claim 5 wherein the family of B7 molecules comprises a molecule selected from the group including B7. 1, B7.2, and B7.3.

5. A method for expressing an increased number of B7 molecules on the surface of an antigen presenting cell to more efficiently potentiate the immune system comprising the steps of:
obtaining an upregulating agent;
administering the upregulating agent to an organism; and, allowing an upregulation of B7 molecules on a cell whereby an expression of the B7 molecules allows reaction with an effector cell, the reaction with the armed effector cell potentiating an immune response.

6. An enhanced macrophage enhanced by immunological response modification, the macrophage enhancing immunological response, comprising a macrophage enhanced by the delivery of a necessary signal that augments an upregulation of a costimulatory molecule, the enhanced upregulation of the costimulatory molecule, in part, caused by a first glucan containing composition interacting with a second glucan containing composition.

7. The macrophage of claim 6 wherein the costimulatory molecule is a B7 molecule.

8. The macrophage of claim 7 wherein the B7 molecule is selected from a group comprising B7.1, B7.2 and B7.3.

9. A beta-glucan preparation which provides a free amino group for conjugation and which can be used as a vaccine adjuvant, comprising:
microparticulate beta-(1,3)-glucan with or without beta-(1,6)-glucan side chains which do not substantially reaggregate upon drying or rehydration;
about 1-10% by weight partially deacetylated N-acetylglucosamine within said beta-glucan that provides a free amino group for vaccine conjugation; and
a vaccine or an antigenic substance, wherein said vaccine or antigenic substance is conjugated with said free amino group.

10. The preparation of claim 9, wherein the glucan contains about 1%-10% by weight chitin or partially deacetylated N-acetylglucosamine.

11. A method of using microparticulate beta-(1,3)-glucan as a vaccine adjuvant comprising the steps of:
preparing or obtaining a microparticulate beta-(1,3)-glucan composition which does not substantially reaggregate upon drying and rehydration which contains partially deacetylated N-acetlyglucosamine with a free amino group;
suspending the microparticulate beta-(1,3)-glucan composition in liquid;
adding at least one vaccine or antigenic substance;
conjugating the vaccine onto the free amino group; and
administering the vaccine to an animal or human.

12. The method of claim 11, wherein the glucan contains less than 5% by weight protein and lipid, more than 85% by weight glucose, and about 1-10% by weight chitin or partially deacetylated N-acetylglucosamine.

13. A vaccine adjuvant which contains microparticulate beta glucan with a free amino group, which enhances the immunologic effects of vaccine or antigenic substance, comprising:
microparticulate beta-(1,3)-glucan with or without beta-(1,6)-glucan side chains which do not substantially reaggregate upon drying or rehydration;
at least 2% by weight partially deacetylated N-acetylglucosamine within said beta-glucan that provides a free amino group for vaccine conjugation.

14. A vaccine conjugate or conjugated antigenic substance attached to the free amino group of microparticulate beta-(1,3)-glucan, which stabilizes the vaccine and enhances the immunologic effects of vaccine, comprising:
microparticulate beta-(1,3)-glucan with or without beta-(1,6)-glucan side chains with about 1-10% by weight partially deacetylated N-acetylglucosamine within said beta-glucan that provides a free amino group for vaccine conjugation which does not substantially reaggregate upon drying or rehydration;
a vaccine or an antigenic substance, wherein said vaccine or antigenic substance is conjugated with said free amino group.

15. A method for preparing a small particle size glucan for dry packaging comprising the steps of:
obtaining a polysaccharide composition comprising the glucan;
hydrating the glucan with a liquid;
disrupting the glucan;
loading the glucan in a sprayer; and,
spraying the glucan.

16. The method of claim 15 further comprising the steps of:
grinding the glucan and
re-hydrating the glucan whereby a portion of the glucan is dissociated into particles of about 1-2 microns in diameter.

17. The method of claim 15 wherein the glucan is substantially glucan selected from the group comprising beta-(1,3)-glucan and beta-(1,6)-glucan.

18. The method of claim 15 wherein the disrupting is accomplished by sonicating the glucan.

19. A method for preparing a small particle size glucan for improved immunological response through enhanced activation of a macrophages and freeze drying the glucan such that re-hydration of the glucan disassociates the glucan, comprising the steps of:
obtaining a polysaccharide composition comprising a glucan containing composition;
hydrating the glucan containing composition with a liquid;
disrupting the glucan;
adding a gelatin solution to the hydrated glucan; and,
freeze drying the glucan.

20. The method of claim 19 further comprising the step of grinding the glucan.

21. The method of claim 19 further comprising the step of rehydrating the glucan whereby a portion of the glucan is dissociated into particles of 0.3-3.0 microns in diameter.

22. The method of claim 19, wherein the disrupting is accomplished by sonicating the glucan.

23. The method of claim 19, wherein the glucan is substantially glucan selected from the group comprising beta-(1,3)-glucan and beta-(1,6)-glucan.
